(19) United States
(12) Patent Application Publication
(10) Pub. No.: US 2008/0280861 A1
(43) Pub. Date: Nov. 13, 2008

(54) Method of Female Contraception and a Kit For Use Therein

(75) Inventors: Herman Jan Tijmen Coelingh Bennink, Noordwijk (NL); Monique Visser, Zeist (NL)

Correspondence Address: THE WEBB LAW FIRM, P.C., 700 KOPPERS BUILDING, 436 SEVENTH AVENUE, PITTSBURGH, PA, 15219, US

(73) Assignee: PANTARHEI BIOSCIENCE B.V., Zeist (NL)

(21) Appl. No.: 11/663,814

(22) PCT Filed: Sep. 27, 2004
(86) PCT No.: PCT/NL04/00668

§ 371 Date: Feb. 28, 2008

(51) Int. Cl. A61K 31/57; A61P 15/18

(52) U.S. Cl. 514/170

(57) ABSTRACT

The invention is concerned with a method of contraception in a female mammal of childbearing capability. The method has two alternating phases—a preservation phase and a shedding phase—and has at least two sequences of (a) a preservation phase of 3-12 months involving continuous oral administration to the female of dosage units containing: (i) an estrogen selected from 17β-estradiol, esters of 17β-estradiol and combinations thereof, in an amount equivalent to a daily oral dosage of 2.2-5 mg 17β-estradiol, and (ii) a progestogen in an amount equivalent to a daily oral dosage of 30-750 μg levonorgestrel; and (b) a shedding phase of 4-12 days during which no progestogen is administered. The invention also relates to a contraceptive kit having one or more packaging units that are separately packaged, individually removable oral dosage units for use in the aforementioned contraceptive method.

### TECHNICAL FIELD OF THE INVENTION

The present invention is concerned with a new method of contraception in mammalian females of childbearing capability. More particularly the present invention relates to such a method comprising orally administering to the female a combination of estrogen and progestogen continuously for at least 3 months.

The invention also relates to a pharmaceutical kit comprising a plurality of oral dosage units, said plurality of daily hormone units containing an estrogen and a progestogen.

### BACKGROUND OF THE INVENTION

Currently on the market there are a number of hormonal contraceptives for females which can be classified into two general types. The first are known as monophasic preparations. These contain a constant amount of an estrogen and a progestogen. Newer preparations known as bi- or triphasic preparations have varying levels of estrogen and progestogen; in most cases consisting of relatively constant levels of estrogen with a step-wise increase in progestogen throughout the cycle. This pattern of estrogen and progestogen administration results in a relatively dominant estrogenic formulation at the beginning of the package with increasing progestogenic activity toward the end of the package. Mono-, bi- and triphasic contraceptives are commonly referred to as combined contraceptives.

Virtually all combined contraceptives have in common that they are based on a regimen which involves an administration-free interval of about 7 days whereby withdrawal bleeding, simulating the natural menses, occurs. Thus 21 day intervals of hormone administration alternate with 7 days during which no hormones are administered.

As an alternative to the aforementioned combined contraceptive methods, the so called sequential method has been proposed. Typical of the sequential contraceptive method is that it comprises two consecutive phases, i.e. one phase during which estrogen and no progestogen is administered and another phase during which a combination of estrogen and progestogen is administered. The first sequential methods, like the aforementioned combined contraceptives, made use of an administration free interval of about 7 days. More recently, sequential methods have been proposed which do not include such an administration-free (or placebo) period, meaning that estrogen is administered throughout the full cycle and that progestogen is co-administered during only part of that cycle. WO 95/17895 (Ehrlich et al.) describes such an uninterrupted sequential method.

Yet another alternative contraceptive method that employs continuous uninterrupted administration of progestogen and at least one estrogen is described in WO 99/12531. In contrast to the aforementioned combined and sequential contraceptive methods, no regular menses occur as the continuous administration of progestogen in the indicated amounts induces amenorrhoea. This so called continuous combined method offers the advantage that it prevents withdrawal bleedings. In addition, the method gives rise to less subjective complaints, such as the symptoms caused by hormone fluctuations, and is associated with a lower risk of VTE than the well-known combined and sequential regimens. Finally, it is believed that the avoidance of chronic fluctuations in blood serum steroid levels may have a positive impact on the occurrence of premenstrual syndrome and the risk of breast cancer.

WO 03/041719 describes a contraceptive method comprising once daily oral administration for a period of 104 days of a combination of 30 μg ethinyl estradiol, 150 mg levonorgestrel and 50 mg dehydroepiandrosterone. The main advantages of such a continuous combined method are said to reside in the prevention of withdrawal bleedings, a decrease of break through bleeding/spotting, less subjective complaints such as the symptoms caused by hormone fluctuations and a lower risk of venous thromboembolism.

### SUMMARY OF THE INVENTION

As mentioned above, a continuous combined contraceptive method that employs a combination of an estrogen and a progestogen is known from WO 99/12531 and WO 03/41719. The present inventors have unexpectedly discovered that significantly less unscheduled bleeding and spotting is observed in such a continuous combined method if (a) the estrogen 17β-estradiol (E2) is employed and administered in a relatively high dosage during a period of 3-12 months and (b) said period is followed by a brief period of 4-12 days during which no progestogen is administered in order to induce menses.

In addition, it was surprisingly found that the prolonged use of a combination of a relatively high dose of E2 and a progestogen very effectively suppresses endometrial thickening. This finding indicates that a continuous combined method employing a relatively high dose of E2 may be used advantageously as a contraceptive method.

It is very unexpected that, despite the use of a high dose of E2, the present method does not give rise to significant endometrial thickening since E2, like other estrogens, is usually associated with endometrial stimulation.

### DETAILED DESCRIPTION OF THE INVENTION

Accordingly, one aspect of the invention is concerned with a method of contraception in a female mammal of childbearing capability, said method consisting of two alternating phases—a preservation phase and a shedding phase—and comprising at least two sequences of:
a. a preservation phase of 3-12 months comprising continuous oral administration to the female of dosage units containing: (i) an estrogen selected from the group consisting of 17β-estradiol, esters of 17β-estradiol and combinations thereof, in an amount equivalent to a daily oral dosage of 2.2-5 mg 17β-estradiol, and (ii) a progestogen in an amount equivalent to a daily oral dosage of 30-750 μg levonorgestrel; and
b. a shedding phase of 4-12 days during which no progestogen is administered.

In accordance with the present invention the dosage units containing the estrogen and/or the progestogen are orally administered at least once weekly, preferably at least once every three days, even more preferably at least once daily and most preferably once daily.

For esters of 17β-estradiol administration in an amount equivalent to a daily oral dosage of a certain amount of 17β-estradiol means administration in an amount equivalent to a daily oral dosage of an amount of said ester that is equimolar to said certain amount of 17β-estradiol. In case of a regime that employs daily oral administration of the estrogen this means that the 17β-estradiol ester is administered in an amount that is equimolar to 2.2-5 mg 17β-estradiol.

The present method may suitable employ any pharmaceutically acceptable substance with sufficient progestogenic activity. The present invention encompasses the use of substances that exhibit progestogenic activity per se as well as esters of such substances. The invention also encompasses the use of progestogen metabolites that exhibit progestogenic activity. Examples of progestogens that may suitably be employed in accordance with the invention include levonorgestrel, dydrogesterone, norethisterone, norgestimate, drospirenone, 3-beta-hydroxydesogestrel, 3-keto desogestrel, 17-deacetyl norgestimate, 19-norprogesterone, acetoxypregnenolone, allylestrenol, anagestone, chlormadinone, cyproterone, demegestone, desogestrel, dienogest, dihydrodydrogesterone, dihydrogesterone, dimethisterone, ethisterone, ethynodiol diacetate, fluorogestone acetate, gastrinon, gestodene, gestrinone, hydroxymethylprogesterone, hydroxyprogesterone, lynestrenol, medrogestone, medroxyprogesterone, megestrol, melengestrol, nomegestrol, norethindrone (=norethisterone), norethynodrel, norgestrel, norgestrienone, normethisterone, progesterone, quingestanol, (17alpha)-17-hydroxy-11-methylene-19-norpregna-4,15-diene-20-yn-3-one, tibolone, trimegestone, algestone acetophenide, nestorone, promegestone, 17-hydroxyprogesterone esters, 19-nor-17hydroxyprogesterone, 17alpha-ethinyl-testosterone, 17alpha-ethinyl-19-nor-testosterone, d-17beta-acetoxy-13beta-ethyl-17alpha-ethinyl-gon-4-en-3-one oxime, esters of these progestogens and combinations thereof. Preferably, the progestogen is selected from the group consisting of levonorgestrel, dydrogesterone, dihydrodydrogesterone, norethisterone, desogestrel, norgestimate, drospirenone, cyproterone, gestodene, trimegestone, progesterone, esters of these progestogens and combinations thereof. Even more preferably, the progestogen is selected from the group consisting of levonorgestrel, dydrogesteron, dihydrodydrogesterone, norethisterone, norgestimate, drosperinone, esters of these progestogens and combinations thereof.

The present method encompasses the use of an ester of 17β-estradiol or an ester of a progestogen. Such esters are capable of liberating 17β-estradiol or a progestogen when used in the present method as a result of metabolic conversion. Examples of suitable esters of 17β-estradiol and progestogens include such substances wherein the hydrogen atom of at least one of the hydroxyl groups has been substituted by an acyl radical of a hydrocarbon carboxylic, sulfonic acid or sulfamic acid of 1-25 carbon atoms; tetrahydrofuranyl; tetrahydropyranal; or a straight or branched chain glycosidic residue containing 1-20 glycosidic units per residue.

Typical examples of esters which can suitably be used in accordance with the invention are esters that can be obtained by reacting the hydroxyl groups of the estrogenic substances with substances that contain one or more carboxy (M⁺⁻OOC—) groups, wherein M⁺ represents a hydrogen or (akali)metal cation. Hence, in a particularly preferred embodiment, the esters are derivatives of 17β-estradiol or a progestogen, wherein the hydrogen atom of at least one of the hydroxyl groups has been substituted by —CO—R, wherein R is a hydrocarbon radical comprising from 1-25 carbon atoms. Preferably R is hydrogen, or an alkyl, alkenyl or aryl radical comprising from 1-20 carbon atoms.

An important advantage of the present method resides in the fact that it suppresses endometrial growth. Typically, in the present method, the continuous daily administration of the combination of estrogen and progestogen is effective in maintaining a substantially constant thin endometrium with a thickness of less than 8 mm, preferably of less than 6 mm.

In order to minimise the risk of breakthrough bleeding the continuous daily administration of progestogen during the preservation phase is interrupted for a period of 4-12 to days, preferably for a period of 5-9 days, once every 3-12 months. These interruptions will cause a predictable withdrawal bleeding (menses), following which continuous administration of the combination of estrogen and progestogen can be resumed with a reduced risk of breakthrough bleeding. In a particularly preferred embodiment during the shedding phase no progestogen and no estrogen is administered.

The withdrawal bleeding during the shedding phase may be induced by simply discontinuing oral administration of dosage units for the duration of the shedding phase. Alternatively, administration of oral dosage units may continue during the shedding phase, with the proviso that the dosage units administered during this phase must not contain progestogen, preferably these units should contain no progestogen and no estrogen. In particular if the present method employs daily oral administration of oral dosage units during the preservation phase, for reasons of compliance it is advantageous to continue daily oral administration of dosage units during the shedding phase.

According to a particularly preferred embodiment the duration of the preservation phase is at least 4 months, more preferably at least 5 months. The risk of unscheduled bleeding and spotting increases if the preservation phase exceeds 9 months. Consequently, in a preferred embodiment, the duration of the preservation phase does not exceed 9 months, more preferably it does not exceed 8 months, most preferably it does not exceed 7 months.

The term “continuous” as used in here, means that the combination of estrogen and progestogen is administered at relatively regular intervals, with no (therapeutically) significant interruptions. Naturally, minor interruptions may occur that do not affect the overall effectiveness of the present method, and indeed such aberrations are encompassed by the present invention. In a preferred embodiment, and more arithmetically, the administration regimen is deemed to be continuous if the longest interval between 2 subsequent administrations is not more than 3.5 times as long as the average interval. Even more preferably said longest interval is not more than 2.5 times, most preferably not more than 1.5 times as long as the average interval.

As explained before, it is a critical element of the present invention to administer a relatively high dose of estrogen. In a particularly preferred embodiment the estrogen is administered in an amount equivalent to a daily oral dosage of at least 2.4 mg 17β-estradiol, more preferably of at least 2.5 mg and most preferably of at least 2.6 mg 17β-estradiol. Preferably, the estrogen is administered in a dosage that does not exceed an amount equivalent to a daily oral dosage of 4.5 mg 17β-estradiol, more preferably it does not exceed an amount equivalent to a daily oral dosage of 4 mg 17β-estradiol and most preferably, it does not exceed an amount equivalent to a daily oral dosage of 3.5 mg 17β-estradiol.

In another preferred embodiment of the present method the progestogen is administered in an amount equivalent to 30-750 μg mg levonorgestrel, more preferably in an amount equivalent to 50-250 μg and most preferably in an amount equivalent to 75-150 μg levonorgestrel. The equivalent dosages for progestogens other than levonorgestrel may be obtained by using conversion factors that are known in the art or that may be established by methods well known to the person skilled in the art. In the table below conversion factors for some progestogens are presented.

| | Conversion | equivalent to 30 μg | | equivalent to 750 μg | |
|---|---|---|---|---|---|
| | factor | levonorgestrel | | levonorgestrel | |
| Levonorgestrel | 1 | 30 | μg | 750 | μg |
| Norethisterone | 7 | 210 | μg | 5.25 | mg |
| Norgestimate | 1.7 | 51 | μg | 1.275 | mg |
| Drospirenone | 20 | 600 | μg | 15 | mg |
| Dydrogesterone | 133 | 4 | mg | 100 | mg |

For esters of progestogens administration in an amount equivalent to a daily oral dosage of a certain amount of levonorgestrel means administration in an amount equivalent to a daily oral dosage of an amount of said ester that is equimolar to the amount of a daily oral dosage of the parent progestogen that is equivalent to said certain amount of levonorgestrel. In case of a regime that employs daily oral administration of the progestogen ester this means that the ester is administered in an amount that is equimolar to the amount of the parent progestogen that is equivalent to 30-750 μg levonorgestrel.

The amount of estrogen administered in accordance with the present method is preferably effective to achieve a 17β-estradiol serum concentration of at least 30 pg/ml, more preferably of at least 50 pg/ml.

The present method is particularly suitable for treating humans, primates, bovines, porcines, equines, canines or felines. Most advantageously the present method is employed in the treatment of humans.

It was found that the present method is particularly advantageous in non-smoking females. Although the inventors do not wish to be bound by theory it is believed that smoking interferes with E2-metabolisation in a way that reduces the bioavailability of E2, e.g. by increasing 2-hydroxylation of E2. Thus, the present method is preferably employed to prevent conception in a non-smoking human female.

It was surprisingly found that the benefits of the present invention may be realised without co-administration of an androgen. Consequently, in a preferred embodiment, the dosage units administered during the preservation phase do not contain an androgen. Even more preferably, also no androgen is administered during the shedding phase.

Another aspect of the present invention concerns a contraceptive kit comprising one or more packaging units, each packaging unit comprising (i) 90-365 separately packaged, individually removable oral dosage units containing an estrogen in an amount equivalent to 2.2-5 mg 17β-estradiol and a progestogen in an amount equivalent to 30-750 μg levonorgestrel, wherein the estrogen is selected from the group consisting of 17β-estradiol, esters of 17β-estradiol and combinations thereof; and (ii) 4-12 separately packaged, individually removable oral dosage units containing no progestogen, preferably containing no progestogen and no estrogen.

Preferably, the estrogen and progestogen are contained within the oral dosage units in amounts that correspond to the preferred daily dosages mentioned herein before.

According to a particularly preferred embodiment of the invention the packaging units contained in the kit are blister packs. Advantageously indicia are provided in the kit, e.g. printed on the packaging unit, that instruct the user to remove the oral dosage units in a particular sequence, thus ensuring that first the oral dosage units containing the estrogen and progestogen are consumed and next the oral dosage units containing no progestogen, or vice versa.

As mentioned herein before, the benefits of the present invention may be realised without co-administration of androgen. Hence, in a preferred embodiment, the oral dosage units containing estrogen and progestogen do not contain an androgen. Even more preferably none of the oral dosage units in the present kit contain an androgen.

In order to ensure that a proper withdrawal bleeding is induced and at the same time the risk of conception is minimised it is advantageous to employ packaging units comprising 5-9 oral dosage units containing no progestogen. In another preferred embodiment, the one or more packaging units comprise 120-240 separately packaged, individually removable oral dosage units containing estrogen and progestogen.

The invention is further illustrated by means of the following examples.

### EXAMPLES

### Example 1

A clinical study was conducted in 30 young healthy female volunteers (15 smokers and 15 non-smokers) who were using a combined monophasic oral contraceptive with at least 30 microgram ethinyl estradiol at the moment of enrolment.

Immediately following 19-25 days of taking this monophasic oral contraceptive, without observing a tablet-free period, treatment with the study medication was commenced. The study medication comprised daily oral administration of dosage units containing 3 mg 17beta-estradiol (E2) and 1.5 mg norethisterone acetate for 3 months (the Preservation phase). Thereupon, volunteers took a daily placebo tablet that contained no E2 and no progestogen for 7 subsequent days (the Shedding phase), resulting in a withdrawal bleeding (menses). The day after the last placebo tablet had been administered daily oral administration of E2 and norethisterone acetate was resumed for another 3 months, again followed by 7 days of placebo administration. Follicular development and endometrial thickness were measured by ultrasonography once every 3 or 4 days depending on follicle growth. Vaginal spotting and bleeding was scored daily by the participants in a diary. In addition, endocrine measurements (E2 and progesterone) were performed in a subgroup of the participants (4 individuals who exhibited follicular growth, 5 randomly selected non-smokers and 5 randomly selected smokers). Specific contraceptive side-effects, mood changes and volunteer appreciation were also evaluated.

No ovulations were observed in any of the participants. In 4 of the 30 participants (2 smokers and 2 non-smokers) persistent follicles of 25-30 mm were seen. In these women, no increase in the progesterone levels was observed. No dominant follicles were seen in the other 26 women (median diameter 5 mm). Endometrial thickness did not change during treatment (median 4.5 mm before and 4.0 mm after treatment). After an initial adaptation period spotting was found to occur infrequently in 3 out of 15 non-smokers. In smokers, however, bleeding and spotting occurred in 3 out of 15 women and spotting only in another 5 women. In some women breast tension, bloating and acne occurred during treatment. No mood changes were registered. Of the 30 participating women, 12 would use this contraceptive when available and 12 would consider its use.

### Example 2

Example 1 is repeated with the exception that the preservation phase comprises daily oral administration for 6 months of dosage units containing 3 mg E2 combined with 150 μg levonorgestrel. Similar results are obtained as described in Example 1.

### Example 3

Example 1 is repeated with the exception that the sequence of preservation phase and shedding phase is repeated three times, the preservation phase comprising daily oral administration for 120 days of dosage units containing 3 mg E2 and 15 mg dydrogesterone. In parallel, another group of 30 women, selected on the basis of the criteria mentioned in example 1, receives daily oral administration for 360 days of dosage units containing 3 mg E2 and 15 mg dydrogesterone. It is found that the incidence of bleeding and spotting in the latter group was significantly higher than in the group that alternately received the combination of E2 and dydrogesterone for 4 months followed by an administration-free interval of 7 days.

1-13. (canceled)

14. A method of contraception in a female mammal of childbearing capability, said method consisting of two alternating phases—a preservation phase and a shedding phase—and comprising at least two sequences of:
a. a preservation phase of 3-12 months comprising continuous oral administration to the female of dosage units containing: (i) an estrogen selected from the group consisting of 17β-estradiol, esters of 17β-estradiol and combinations thereof, in an amount equivalent to a daily oral dosage of 2.2-5 mg 17β-estradiol, and (ii) a progestogen in an amount equivalent to a daily oral dosage of 30-750 μg levonorgestrel; and
b. a shedding phase of 4-12 days during which no progestogen and no estrogen is administered.

15. The method according to claim 14, wherein the preservation phase covers 4-8 months.

16. The method according to claim 14, wherein the preservation phase comprises daily oral administration of the dosage units containing the estrogen and the progestogen.

17. The method according to claim 14, wherein the shedding phase comprises daily oral administration of dosage units containing no progestogen and no estrogen.

18. The method according to claim 14, wherein the dosage units administered during the preservation phase do not contain androgen.

19. The method according to claim 14, wherein the progestogen is selected from the group consisting of dydrogesterone, norethisterone, levonorgestrel, norgestimate, drospirenone, 3-beta-hydroxydesogestrel, 3-keto desogestrel, 17-deacetyl norgestimate, 19-norprogesterone, acetoxypregnenolone, allylestrenol, anagestone, chlormadinone, cyproterone, demegestone, desogestrel, dienogest, dihydrodydrogesterone, dihydrogesterone, dimethisterone, ethisterone, ethynodiol diacetate, fluorogestone acetate, gastrinon, gestodene, gestrinone, hydroxymethylprogesterone, hydroxyprogesterone, lynestrenol, medrogestone, medroxyprogesterone, megestrol, melengestrol, nomegestrol, norethindrone (=norethisterone), norethynodrel, norgestrel, norgestrienone, normethisterone, progesterone, quingestanol, (17alpha)-17-hydroxy-11-methylene-19-norpregna-4,15-diene-20-yn-3-one, tibolone, trimegestone, algestone acetophenide, nestorone, promegestone, 17-hydroxyprogesterone esters, 19-nor-17hydroxyprogesterone, 17alpha-ethinyl-estosterone, 17alpha-ethinyl-19-nor-testosterone, d-17beta-acetoxy-13beta-ethyl-17alpha-ethinyl-gon-4-en-3-one oxime, esters of these progestogens and combinations thereof.

20. The method according to claim 14, wherein the estrogen is administered in an amount equivalent to a daily oral dosage of 2.4-4 mg 17β-estradiol.

21. The method according to claim 20, wherein the estrogen is administered in an amount equivalent to a daily oral dosage of 2.5-3.5 mg 17β-estradiol.

22. A contraceptive kit comprising one or more packaging units, each packaging unit comprising (i) 90-365 separately packages, individually removable oral dosage units containing an estrogen in an amount equivalent to 2.2-5 mg 17β-estradiol and a progestogen in an amount equivalent to 30-750 μg levonorgestrel, wherein the estrogen is selected from the group consisting of 17β-estradiol, esters of 17β-estradiol and combinations thereof; and (ii) 4-12 separately packaged, individually removable oral dosage units containing no progestogen and no estrogen.

23. The kit according to claim 22, wherein the one or more packaging units are blister packs.

24. The kit according to claim 22, wherein the oral dosage units containing estrogen and progestrogen do not contain androgen.

25. The kit according to claim 22, wherein the one or more packaging units comprise 120-240 separately packaged, individually removable oral dosage units containing estrogen and progestogen.
